(19) United States
(12) Patent Application Publication
(10) Pub. No.: US 2025/0019752 A1
(43) Pub. Date: Jan. 16, 2025

(54) COMPOSITIONS AND METHODS FOR REDUCING MASTER MIX CONTAMINATION

(71) Applicant: Life Technologies Corporation, Carlsbad, CA (US)

(72) Inventors: Daniel Philippe Blanchard, South San Francisco, CA (US); Maria Cinta Gomez Silvan, Burlingame, CA (US)

(21) Appl. No.: 18/706,621

(22) PCT Filed: Nov. 2, 2022
(86) PCT No.: PCT/US2022/048717

§ 371 Date: May 1, 2024

Related U.S. Application Data
(60) Provisional application No. 63/274,656, filed on Nov. 2, 2021.

(51) Int. Cl. C12Q 1/6848; C12Q 1/686

(52) U.S. Cl. CPC C12Q 1/6848; C12Q 1/686

(57) ABSTRACT

This disclosure describes master mix compositions, nucleic acid amplification kits, methods of manufacturing master mix compositions, and methods of using master mix compositions that minimize or eliminate the negative effects of contaminant nucleic acids. Conventional master mix compositions often include contaminant nucleic acids.

### CROSS-REFERENCE TO RELATED APPLICATIONS

This application claims priority to and the benefit of U.S. Provisional Patent Application No. 63/274,656, filed Nov. 2, 2021, which is incorporated herein by reference in its entirety.

### BACKGROUND

This disclosure relates to compositions, kits, and methods for the amplification and/or detection of nucleic acids.

For many medical, diagnostic, and forensic applications, amplification of a particular nucleic acid sequence is essential to allow its detection in, or isolation from, a sample in which it is present in very low amounts.

Polymerase chain reaction (PCR) is an in vitro method for the enzymatic synthesis of specific DNA sequences using two oligonucleotide primers that hybridize to opposite strands and flank the region of interest in target DNA. A repetitive series of reaction steps involving template denaturation, primer annealing, and the extension of the annealed primers by DNA polymerase results in the exponential accumulation of a specific fragment whose termini are defined by the primers. PCR can selectively enrich a specific DNA sequence by several orders of magnitude.

PCR “master mixes” improve the efficiency of amplification reactions. These master mixes contain a combination of reagents common to most PCR reactions such as a buffer, a salt such as MgCl₂, deoxynucleotide triphosphates (dNTPs), and a DNA polymerase. When performing PCR, the reaction volume includes the master mix and a primer pair specific for a target nucleic acid. Typically, master mixes are manufactured and distributed as concentrated solutions or lyophilized powders which are subsequently diluted or dissolved when final reaction volumes are assembled.

There is an ongoing need for improved master mix compositions, and there is an ongoing need, in particular, for master mix compositions capable of minimizing or eliminating the effects of nucleic acid contamination in amplification reactions.

### BRIEF DESCRIPTION OF THE DRAWINGS

FIG. 1 provides an overview of an example process for treating a master mix composition to reduce the effects of contaminant template nucleic acid.

FIG. 2 illustrates the effects of an intercalating agent (propidium monoazide (PMA) in this example), which is capable of intercalating with contaminant DNA, and which is activated by light to covalently bond to DNA and thereby prevent the DNA from being used as a template in amplification reactions.

FIG. 3 illustrates the effects the intercalating agent (PMA, in this example) can have on fluorescent signals of the reaction volume in which the PMA is present.

FIGS. 4A and 4B illustrate quantitative PCR (qPCR) amplification plots for a pan-bacterial (16S) assay comparing amplification of the no-template control (NTC) of a conventional master mix (FIG. 4A) to amplification of the NTC of the master mix composition prepared according to one embodiment (FIG. 4B), with the NTC of the conventional master mix showing higher background signal as compared to the NTC of the presently disclosed master mix.

FIG. 5 illustrates the amplification signal of both a conventional master mix and the master mix prepared according to one embodiment over various amounts of spiked-in bacterial genomes (0, 1, 10, 100, 1,000, 10,000, and 100,000 genomes per reaction) using a pan-bacterial (16S) assay, showing that the conventional master mix obscures any signal beneath the contamination level and showing that the presently disclosed master mix (‘Improved MMx’ from the figure) enables a lower limit of detection (LOD) and expands the dynamic range of the assay.

FIG. 6 illustrates the fluorescence signals of various dyes during an amplification reaction, showing that a master mix composition including 10 μM of PMA reduces the FAM signal as compared to an otherwise similar master mix composition that lacks PMA.

FIG. 7A illustrates effects of autoclaving fish skin gelatin using different autoclaving protocols, showing that a pan-bacterial (16S) qPCR assay detected similar levels of contaminant DNA in a master mix composition elaborated with untreated fish gelatin (fish skin gelatin, no autoclaving) and the master mix composition elaborated with fish gelatin that was autoclaved for 20 minutes, and showing substantial lack of contamination in the negative control (no fish skin gelatin) and the master mix composition elaborated with fish gelatin that was autoclaved for 80 minutes.

FIG. 7B illustrates the effects of autoclaving the fish skin gelatin on the ability of the master mix compositions composed of fish skin gelatin to provide inhibitor tolerance in an assay with spike-in of known amounts of a XENO sequence, showing that at a concentration of 80 ng/μl of a humic acid inhibitor, the composition receiving the 80 minute autoclaved fish gelatin performed substantially the same as the composition receiving the 20 minute autoclaved fish gelatin and the composition receiving untreated fish gelatin, and showing that at a concentration of 100 ng/μl of humic acid, the composition receiving the 80 minute autoclaved fish gelatin performed substantially the same as the composition receiving the 20 minute autoclaved fish gelatin.

FIG. 8 illustrates results of various quality control screening qPCR assays using the master mix compositions prepared according to some embodiments, showing that in all qPCR assays tested, the presently disclosed master mix compositions performed as expected for known amounts of spiked-in nucleic acid and showed no detectable contamination.

### DETAILED DESCRIPTION

Disclosed herein are master mix compositions, nucleic acid amplification kits, methods of manufacturing master mix compositions, and methods of using master mix compositions that minimize or eliminate the negative effects of contaminant nucleic acids. Conventional master mix compositions often include contaminant nucleic acids. When a nucleic acid amplification reaction (e.g., PCR) is performed using such contaminated master mix compositions, the contaminant nucleic acids can act as templates, using up a portion of the primers, dNTPs, and polymerase activity intended for the targeted nucleic acids of the assay.

For example, a “pan” assay, such as a pan-bacteria assay targeting the 16S rRNA gene, will also amplify contaminant bacterial DNA in the master mix. This results in a baseline signal caused by the contaminant DNA. As a result, the LOD is increased because it takes more target DNA in the sample just to overcome the baseline signal associated with the contaminant DNA. In effect, the contaminant DNA obscures any signal under the level of contamination. Eliminating or otherwise neutralizing the ability of the contaminant DNA to participate in the amplification reaction can therefore lower the LOD and expand the dynamic range of an assay.

Other types of nucleic acid contaminants can be present and can affect assays with other targets. For example, master mix compositions may include contaminant nucleic acids from one or more bacterial strains or taxa, from one or more fungal strains or taxa, from one or more viruses, from one or more eukaryotic sources, from one or more vertebrate sources, from human sources, or combination thereof. If an assay is designed to target a nucleic acid that is also present in the contaminant nucleic acids, the contaminant nucleic acids can limit the effectiveness of the assay.

The embodiments disclosed herein beneficially provide master mix compositions that minimize or eliminate the effects of contaminant nucleic acid. Accordingly, assays utilizing such master mix compositions are capable of improved LOD and expanded dynamic range as compared to assays that utilize conventional master mix compositions.

### Overview of Methods of Manufacturing Master Mix Compositions

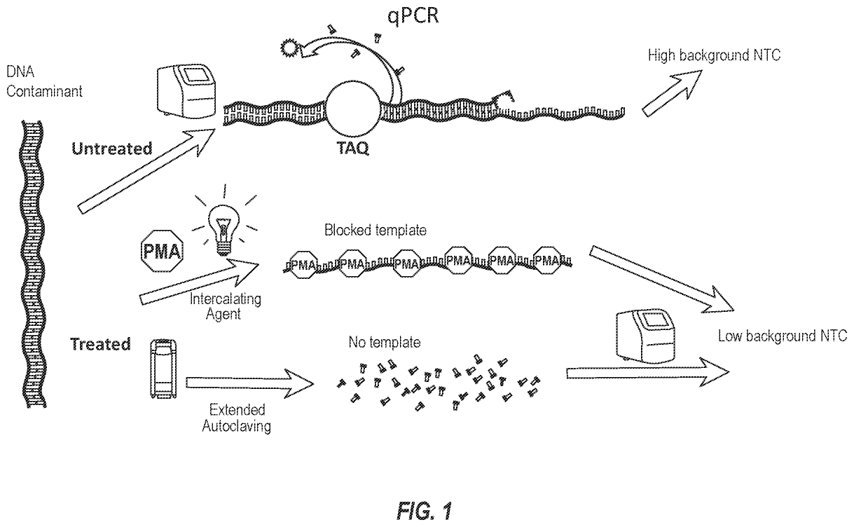

FIG. 1 provides an overview of an example process for treating a master mix composition to reduce the effects of contaminant DNA. As shown, where a master mix composition is untreated, contaminant DNA present within the master mix can serve as a template for amplification during the reaction. In this example, Taq polymerase causes the release of a fluorescent label upon extension of the contaminant template, raising the baseline signal of the NTC. Because of the raised background signal, any target nucleic acid within the sample must be present in amounts sufficiently above the contamination level to be detectable.

FIG. 1 also schematically illustrates a method of treating a master mix composition to reduce the effect of contaminant nucleic acids. As shown, the method may include one or both of (i) treatment with an intercalating agent to block contaminant nucleic acids from use as templates during amplification, and (ii) an extended autoclaving protocol that destroys contaminant nucleic acids. The treatment method lowers or eliminates the NTC background based on nucleic acid contamination of the master mix. In the illustrated example, PMA is utilized as an intercalating agent. As described in more detail below, other intercalating agents may additionally or alternatively be utilized.

During manufacture, a mixture may be treated by adding the intercalating agent at a concentration of at least about 200 μM, at least about 180 μM, at least about 160 μM, at least about 140 μM, at least about 120 μM, at least about 100 μM, or at least about 90 μM, or at least about 80 μM, or at least about 70 μM, or at least about 60 μM, or at least about 50 μM, or at least about 40 μM, or at least about 30 μM, or at least about 20 μM, or at least about 10 μM, or at least about 5 μM, or at least about 4 μM, or at least about 3 μM, or at least about 2 μM, or at least about 1 μM, or at least about 100 nM. Upon activation with light of appropriate wavelength, the intercalating agent such as PMA that has intercalated with contaminant DNA covalently bonds with the DNA and renders it incapable of acting as a template for amplification. The treated mixture may then be combined with other components to form the final master mix composition.

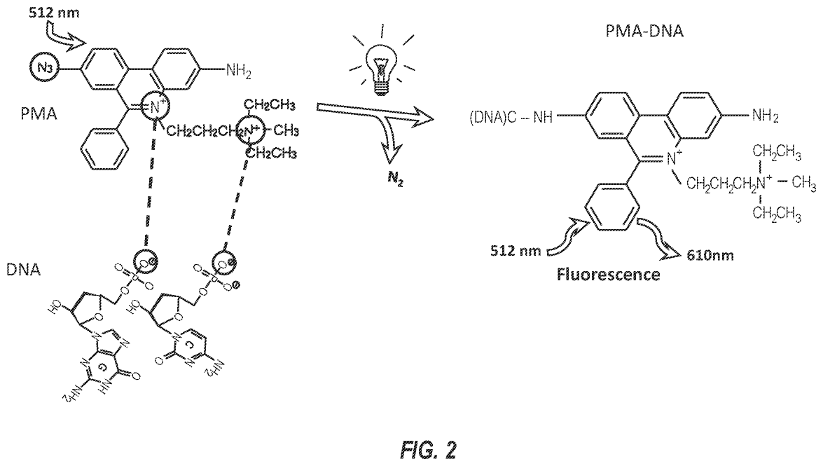

FIG. 2 illustrates the effects of an intercalating agent (PMA in this example), which is capable of intercalating contaminant DNA, and which is activated by light to covalently bond to DNA and thereby prevent the DNA from being used as a template in amplification reactions. As shown, the PMA includes charged N groups that support the intercalation of the PMA with the DNA molecule. Upon activation using light with a wavelength of about 512 nm (e.g., about 475 nm to about 550 nm), the aromatic phenanthridine absorbs the light, and the azide group loses an N2 and reacts with the DNA molecule to form an amine bond. The resulting PMA-DNA molecule therefore includes propidium (i.e., post-activated PMA) bonded via an amine bond to DNA.

Referring to FIG. 1, the illustrated example also includes an extended autoclaving process in which one or more of the master mix components are subjected to autoclaving for a period that is longer than typical autoclaving. In some embodiments, the extended autoclaving is performed at a temperature of about 100° C. to about 150° C. and a pressure of about 60-140 kPa (about 9-20 psi), or at a temperature of about 105° C. to about 140° C. and a pressure of about 70-130 kPa (about 10-19 psi), or at a temperature of about 110° C. to about 130° C. and a pressure of about 80-120 kPa (about 12-17 psi), or at a temperature of about 115° C. to about 125° C. and a pressure of about 90-110 kPa (about 13-16 psi), or at a temperature of about 121° C. and a pressure of about 100-105 kPa (about 15 psi). In some embodiments, the extended autoclaving is performed for a at least about 40 minutes, or at least about 50 minutes, or at least about 60 minutes, or at least about 70 minutes, or at least about 80 minutes, or at least 90 minutes, or at least 100 minutes, or at least 120 minutes, or at least 200 minutes.

In some embodiments, the master mix composition is manufactured by providing a first mixture that comprises one or more nucleic acid amplification components and adding the intercalating agent to the first mixture. The first mixture may then be exposed to light to activate the intercalating agent and cause covalent bonding between the intercalating agent and contaminant DNA present in the first mixture. In some embodiments, the method may further comprise providing a second mixture that comprises one or more nucleic acid amplification components and subjecting the second mixture to extended autoclaving as described herein. The first and second mixtures may then be combined. In some embodiments, one or more additional component and/or mixture may be combined with the first and/or second mixtures to form a master mix. In some embodiments, at least some of the additional component(s) and/or mixture(s) may not be treated with an intercalating agent or undergo an extended autoclaving. In some embodiments, the additional component(s) and/or mixture(s) may be pre-tested for lack of nucleic acid contamination.

Providing a first mixture and a second mixture in this manner allows for the treatment of a first subset of components of the master mix with the intercalating agent and the treatment of a second, different subset of the components of the master mix with extended autoclaving. This enables optimal use of the separate treatments to enhance the effectiveness of the resulting master mix composition. As described in more detail below, excessive intercalating agent in the assembled master mix composition can interfere with fluorescent signals when used with assays relying on fluorescent label detection. On the other hand, certain master mix components are not amenable to the harsh conditions of autoclaving. Thus, it is beneficial to treat some master mix components with extended autoclaving, and other master mix components with an intercalating agent, so that in combination the two treatment prongs enable effective removal or inactivation of contaminant nucleic acids without introducing other limitations to the amplification reaction. Some embodiments may include additional components and/or mixtures that are neither treated with an intercalating agent nor subjected to extended autoclaving. In some embodiments, for example, the additional component(s) and/or mixture(s) may be sourced and known to be free of nucleic acid. In some embodiments, for example, the additional component(s) and/or mixture(s) may be pre-tested for lack of nucleic acid contamination.

In some embodiments, the master mix composition is manufactured by providing a mixture that comprises one or more nucleic acid amplification components and adding the intercalating agent to this mixture without combining with a mixture subjected to extended autoclaving. In other embodiments, the master mix composition is manufactured by providing a mixture that comprises one or more nucleic acid amplification components subjected to extended autoclaving but without combining with a mixture that was treated with the intercalating agent. Therefore, in some embodiments, only one of the steps comprising (i) treatment of intercalating agent and (ii) extended autoclaving may be applied to manufacture a master mix composition.

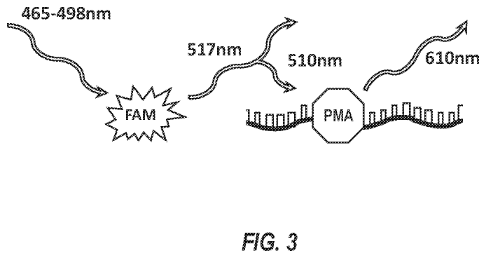

FIG. 3 illustrates the effects the intercalating agent (PMA, in this example) can have on fluorescent signals of the reaction volume in which the PMA is present. As shown, fluorescein, 5-carboxyfluorescein (FAM) absorbs light at about 465-498 nm and emits a signal at about 517 nm. However, because bonded PMA has an absorption maximum of about 510-512 nm, a portion of the FAM signal can be absorbed by the bonded PMA, which emits at a wavelength of about 610 nm. As illustrated, excessive levels of intercalating agent in the final master mix can detrimentally affect the signals of one or more dyes. Although FAM and PMA are illustrated here, other dye signals can additionally or alternatively be affected by PMA and/or other intercalating agents. The intercalating agent can unwantedly absorb light within a target dye channel and/or emit light too close to another dye channel.

Accordingly, preferred embodiments limit the concentration of intercalating agent in the final master mix composition to no more than about 100 μM, or no more than about 75 μM, or no more than about 50 μM, or no more than about 25 μM, or no more than about 10 μM, or no more than about 7.5 μM, or no more than about 5 μM, or no more than about 2.5 μM, or no more than about 1 μM, or no more than about 750 nM, or no more than about 500 nM, or no more than about 250 nM, no more than about 100 nM, no more than about 50 nM, no more than about 25 nM, no more than about 10 nM, no more than about 5 nM, or no more than about 1 nM, or no more than about 0.1 nM.

In some embodiments, the one or more nucleic acid amplification components treated with the intercalating agent (i.e., components in the “first mixture”) include components that are not amenable to autoclaving. For example, the set of components subjected to treatment with the intercalating agent may include one or more proteins (e.g., enzymes) such as DNA polymerase, a DNA polymerase antibody, reverse transcriptase (e.g., in RT-PCR applications), inhibitor tolerance agent (e.g., bovine serum albumin (BSA)), and combinations thereof.

In some embodiments, the one or more nucleic acid amplification components treated with extended autoclaving (i.e., components in the “second mixture”) include all the master mix components not treated with the intercalating agent. In some embodiments, the components treated with extended autoclaving include one or more buffers, salts, water, glycerin, other carrier components, inhibitor tolerance agents, and combinations thereof. In some embodiments, the components treated with extended autoclaving include a gelatin, such as fish skin gelatin. When utilized the inhibitor tolerance agent may include gelatin (e.g., bovine), serum albumin (e.g., bovine), polyethylene glycol (PEG), resin, agarose, other inhibitor tolerance agents known in the art, or combinations thereof.

### Intercalating Agents

Whenever molecular formulas or components thereof are described herein, it will be understood that moieties and groups of the disclosed formulae may be substituted or unsubstituted. Thus, except where specified otherwise, disclosed moieties and groups may be substituted or unsubstituted. As an example, a moiety or group may include one or more substituents independently selected from alkyl, aromatic, phenyl, halogen, alkyl, amine, amino, alkylamino, and dialkylamino. It will also be understood that the intercalating agents described herein may be provided in suitable salt forms, such as bromide or iodide salts.

In some embodiments, the intercalating agent has a formula as in Formula I.

X—Ar—R₁

wherein
X comprises an azide, a charged N, or an amine, or an amino, wherein when X comprises an amino/amine, the amino/amine is NH₂, NR₂H, or NR₂R₃, wherein R₂ and R₃ are independently a DNA molecule to which the intercalating agent is covalently bonded or a group comprising an alkyl,
Ar is a heterocyclic aromatic group with a ring structure having at least one charged N, and
R₁ is attached to the charged N of the Ar and comprises an alkyl.

As discussed above, the intercalating agent may be added to one or more of the master mix components and be activated to covalently bond to contaminant DNA. In its pre-activated form, the intercalating therefore includes a moiety (such as an azide) capable of forming a covalent bond with the contaminant DNA upon activation. In some embodiments, a majority of, or substantially all of, the pre-activated intercalating agent (i.e., prior to light treatment to initiate covalent bonding) includes an azide group. In some embodiments, at least about 60%, or at least about 70%, or at least about 80%, or at least about 85%, or at least about 90%, or at least about 95%, or at least about 97% of the pre-activated intercalating agent is in the azide-including form.

The aromatic group enables the intercalating agent to absorb light and therefore enables activation and bonding to the DNA molecule. The charged N of the aromatic group promotes association and intercalation between the intercalating agent and the DNA molecule. R₁ may also include an amino group, an amine group, or charged N to further promote association and intercalation between the intercalating agent and the DNA molecule; however, R₁ does not necessarily require an amino group, an amine group, or charged N for effective intercalation to occur.

In some embodiments, substantially all of the intercalating agent in a final master mix composition will be in the post-activated form. In other words, with reference to Formula I, X will primarily be a free amino group (for post-activated but unbound intercalating agent) or will form a secondary or tertiary amine bond with a DNA molecule. However, in some embodiments, amounts (e.g., trace amounts) of pre-activated intercalating agent may remain. For example, a portion of the intercalating agent may have the form of Formula I when X is an azide.

With reference to Formula I, in some embodiments, R₁ comprises a charged N, amine and/or amino. In some embodiments, the charged N of R₁ is a quaternary ammonium. In some embodiments, the quaternary ammonium is attached to the Ar by a C₁-C₆ alkyl. In some embodiments, the quaternary ammonium is attached to the Ar by a C₂-C₃ alkyl. In some embodiments, the quaternary ammonium is bonded to three C₁-C₂ alkyl groups. For example, in some embodiments, R₁ has a structure according to Formula IIa or IIb:

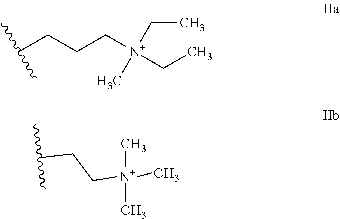

With reference to Formula I, in some embodiments, the ring structure of Ar has two or three rings. For example, Ar may be a benzopyridine or dibenzopyridine. More particularly, Ar may be a phenanthridine, quinoline, isoquinoline, acridine, or aminoacridine.

Ar may be substituted or unsubstituted. In embodiments where Ar is substituted, the substituents of the Ar are independently selected from the group consisting of phenyl, halogen, alkyl, amine, amino, alkylamino, and dialkylamino. In some embodiments, least one of the substituents is phenyl. For example, the at least one phenyl group may be connected to the Ar at a carbon that is adjacent the charged N of the Ar. In some embodiments at least one of the substituents is an amino group (e.g., NH₂).

In some embodiments, Ar is a phenanthridine, R₁ has a structure according to Formula IIa or IIb, and the Ar includes a phenyl substituent and an amino substituent. For example, in some embodiments the intercalating agent comprises 3-Amino-5-[3-(methyldiethylaminio)propyl]-6-phenyl-8-azidophenanthridinium, commonly referred to simply as propidium monoazide (PMA), which has a structure according to Formula III:

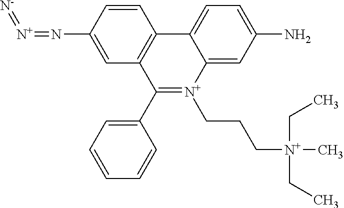 [SMILES: CC[N+](C)(CC)CCC[n+]1c(-c2ccccc2)c2cc(N=[N+]=[N-])ccc2c2ccc(N)cc21]

In some embodiments, Ar is a phenanthridine, R₁ is an ethyl, and the Ar includes a phenyl substituent and an amino substituent. For example, in some embodiments the intercalating agent comprises 3-Amino-8-azido-5-ethyl-6-phenylphenanthridinium, commonly referred to simply as ethidium monoazide (EMA), which has a structure according to Formula IV:

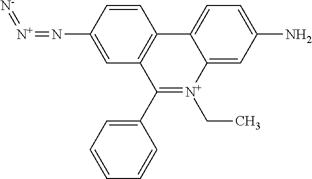 [SMILES: CC[n+]1c(-c2ccccc2)c2cc(N=[N+]=[N-])ccc2c2ccc(N)cc21]

In some embodiments, Ar is a quinoline and R₁ has a structure according to Formula IIa or IIb. For example, in some embodiments the intercalating agent comprises 6-azido-l-(3-(trimethylammonio)propyl)quinolin-1-ium, which has a structure according to Formula V:

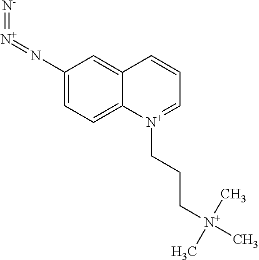 [SMILES: C[N+](C)(C)CCC[n+]1cccc2cc(N=[N+]=[N-])ccc21]

In any of the compounds of Formulas III-V, the azide can be converted to a charged N, an amino group, or an amine bonded (secondary or tertiary) DNA molecule.

### Additional Master Mix Composition Components & Details

In some embodiments, a master mix composition is provided as a concentrated stock solution intended for mixing with other components of the reaction (e.g., the sample itself, other optional buffers, nuclease free water, etc.) to form the full reaction mixture. In such embodiments, the component amounts disclosed herein can therefore be understood to refer to a concentrated stock solution intended for dilution in the resulting reaction mixture. The stock solution may be formulated as a 2×, 3×, 4×, 5×, 6×, 7×, 8×, 9×, or 10× solution, for example.

In some embodiments, the polymerase is a thermostable DNA polymerase, such as a Taq DNA polymerase, a mutant, variant, or derivative thereof. Various polymerases are known to the person skilled in the art and may be utilized accordingly. In some embodiments, the master mix also includes a reverse transcriptase to enable reverse transcription of target RNA to DNA or cDNA. The RNA target may include, for example, mRNA, miRNA or other types of small RNA, viral RNA targets, or other RNA targets.

In some embodiments, the master mix also includes one or more deoxynucleotide triphosphates (dNTPs). The master mix may include one or more of deoxythymidine triphosphate (dTTP), deoxyadenosine triphosphate (dATP), deoxycytidine triphosphate (dCTP), deoxyguanosine triphosphate (dGTP), 7-deaza-dGTP, or deoxyuridine triphosphate (dUTP). The master mix may additionally or alternatively include nucleotide analogs such as unnatural nucleotides, mutagen nucleotides, and/or fluorophore-labelled nucleotides. Each provided nucleotide may have a concentration of about 0.5 mM to about 5 mM, for example. The concentration of the individual dNTPs need not be identical. For example, dATP, dCTP, dGTP may be present in the composition at a first concentration while dUTP is present in the composition at a second, higher concentration.

In some embodiments, the master mix also includes a carrier. The carrier may include any formulation suitable for carrying the other components without significantly interfering with the intended amplification process. Examples include water, standard PCR buffer components, and polyols. In some embodiments, the carrier includes glycerol. The glycerol may be included at a concentration of about 1% to about 65% (v/v), or about 5% to about 65% (v/v), or about 10% to about 60% (v/v), or about 20% to about 55% (v/v), or about 35% to about 50% (v/v), or within a range having endpoints defined by any two of the foregoing values.

The master mix compositions described herein may also include other standard master mix components, such as buffer agents and/or salt solutions to provide appropriate pH and ionic conditions to maintain stability and effectiveness of the included polymerase enzymes. Examples of salt solutions include, for example, potassium chloride, potassium acetate, potassium sulfate, ammonium sulfate, ammonium chloride, ammonium acetate, magnesium chloride, magnesium acetate, magnesium sulfate manganese chloride, manganese acetate, manganese sulfate, sodium chloride, sodium acetate, lithium chloride and lithium acetate. It is to be understood that a wide variety of buffers and salt solutions are known in the art beyond those specifically disclosed herein.

In some embodiments, the master mix includes one or more components to enable enzymatic degradation of pre-existing PCR products. For example, the master mix may include heat labile uracil-DNA glycosylase (Heat labile UDG). Such components can be generated or selected using processes known in the art. Alternatively, a commercially available UDG can be used.

In some embodiments, the master mix includes one or more components to enable “hot start” PCR. For example, the master mix may include an antibody, an aptamer, a hairpin primer, or a sequestration wax bead. Such components can be generated or selected using processes known in the art. Alternatively, a commercially available antibody can be used, for example, the TaqStart Antibody (Clontech) which is effective with any Taq-derived DNA polymerase, including native, recombinant, and N-terminal deletion mutants.

### Nucleic Acid Amplification Kits

Some embodiments are directed to nucleic acid amplification kits comprising a master mix composition as described herein and at least one pair of primers. In some embodiments a kit further includes one or more probes. The one or more probes and/or one or more of the primers may include a detectable label.

The master mix compositions described herein can be packaged in a suitable container capable of holding the composition and which will not significantly interact with components of the composition. The container can be one designed to permit easy dispensing of the composition by individuals or by a liquid handling instrument. The containers of composition can be further packaged into multi-pack units.

In some embodiments, the primers are used in nucleic acid assays at a concentration from about 100 nM to 1 mM (e.g., 300 nM, 400 nM, 500 nM, etc.), including intervening concentration amounts and ranges defined by endpoints selected from any two of the foregoing values. In some embodiments, probes described herein are also used in a nucleic acid assay and are provided at a concentration from about 50 nM to 500 nM (e.g., 75 nM, 125 nM, 250 nM, etc.), including intervening concentration amounts and ranges defined by endpoints selected from any two of the foregoing values.

Various probes are known in the art, for example TaqMan® probes (see, e.g., U.S. Pat. No. 5,538,848) various stem-loop molecular beacons (see, e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion probes (see, e.g., Solinas et al., 2001, Nucleic Acids Research 29:E96 and U.S. Pat. No. 6,589,743), bulge loop probes (see, e.g., U.S. Pat. No. 6,590,091), pseudo knot probes (see, e.g., U.S. Pat. No. 6,589,250), cyclicons (see, e.g., U.S. Pat. No. 6,383,752), MGB Eclipse probe (Epoch Biosciences), hairpin probes (see, e.g., U.S. Pat. No. 6,596,490), peptide nucleic acid (PNA) light-up probes, self-assembled nanoparticle probes, and ferrocene-modified probes described, for example, in U.S. Pat. No. 6,485,901 and Mhlanga et al., 2001, Methods 25:463-471. Probes can also comprise two probes, wherein for example a fluor is on one probe, and a quencher on the other, wherein hybridization of the two probes together on a target quenches the signal, or wherein hybridization on a target alters the signal signature via a change in fluorescence.

Exemplary detectable labels (e.g., for a probe and/or primer) include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy,fluorescein (JOE)); 6-carboxy-1,4-dichloro-2′,7′-dichloro,fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet)), red fluorescent proteins (e.g., RFP, mCherry), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl¬rhodamine), Tetramethylrhodamine (TRITC), WT), Texas Red, Texas Red-X, among others as known to those of skill in the art.

Detector probes may be associated with quenchers such as dark fluorescent quencher (DFQ), black hole quenchers (BHQ), Iowa Black, QSY quencher, Dabsyl and Dabcel sulfonate/carboxylate quenchers, and MGB-NFQ quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with S03 instead of the carboxylate group, phosphoramidite forms of fluorescein, and/or phosphoramidite forms of Cy5, for example.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30(9): e37), Hayashi et al. (Nucleic Acids Res. 1989 May 11; 17(9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 Jul: 10.2144/btn-2020-0057).

In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product.

### Nucleic Acid Amplification

In some embodiments, a method for amplifying a nucleic acid comprises: providing a master mix composition as described herein; mixing the master mix composition with a pair of primers configured to hybridize with a target nucleic acid; and subjecting the master mix composition and primers to amplification conditions to enable amplification of the target nucleic acid.

Amplified products (“amplicons”) resulting from use of one or more embodiments described herein can be generated, detected, and/or analyzed using any suitable method and on any suitable platform. In some embodiments, the nucleic acid targets may be single-stranded, double-stranded, or any other nucleic acid molecule of any size or conformation. The nucleic acid assays described herein can include PCR assays (see, e.g., U.S. Pat. No. 4,683,202), loop-mediated isothermal amplification (“LAMP”) (see, e.g., U.S. Pat. No. 6,410,278), and other methods described herein for detecting nucleic acid targets in a sample. In some embodiments, the PCR is real time or quantitative PCR (qPCR). In some embodiments, the PCR is an end point PCR. In some embodiments, the PCR is digital PCR (dPCR).

In some embodiments, the method is RT-PCR and further includes subjecting the target nucleic acid to a reverse transcription reaction prior to amplification via PCR. In some embodiments, the master mix composition includes at least one reverse transcriptase, and the amplification method includes RT-PCR. In some embodiments, the amplification method includes one-step RT-PCR (e.g., in a single vessel or reaction volume) in which one or more reverse transcriptases are used in combination with one or more DNA polymerases.

Amplification methods may be singleplex or multiplex. In some embodiments, each dye is associated with one or more target sequences. In some embodiments, up to 2, 4, 6, 8, 10, or 12 targets are amplified and tracked real-time within a single reaction vessel, using any combination of detectable labels disclosed herein or otherwise known to those of skill in the art. In some multiplex embodiments, at least one of the targets is an endogenous or exogenous internal positive control (e.g., RNase P).

In general, PCR thermal cycling includes an initial denaturing step at high temperature, followed by a repetitive series of temperature cycles designed to allow template denaturation, primer annealing, and extension of the annealed primers by the polymerase. Generally, the samples are heated initially for about 2 to 10 minutes at a temperature of about 95° C. to denature the double stranded DNA sample. Then, in the beginning of each cycle, the samples are denatured for about 10 to 60 seconds, depending on the samples and the type of instrument used. After denaturing, the primers are allowed to anneal to the target DNA at a lower temperature, from about 40° C. to about 65° C. for about 10 to 60 seconds. Extension of the primers by the polymerase is often carried out at a temperature ranging from about 60° C. to about 72° C. The amount of time used for extension will depend on the size of the amplicon and the type of enzymes used for amplification and is readily determined by routine experimentation. Additionally, the annealing step can be combined with the extension step, resulting in a two-step cycling. Thermal cycling may also include additional temperature shifts in PCR assays. The number of cycles used in the assay depends on many factors, including the primers used, the amount of sample DNA present, and the thermal cycling conditions. The number of cycles to be used in any assay may be readily determined by one skilled in the art using routine experimentation. Optionally, a final extension step may be added after the completion of thermal cycling to ensure synthesis of all amplification products.

### Abbreviated List of Terms & Definitions

As used herein, “nucleic acid” includes compounds having a plurality of natural nucleotides and/or non-natural (or “derivative”) nucleotide units. A “nucleic acid” can further comprise non-nucleotide units, for example peptides. “Nucleic acid” therefore encompasses compounds such as DNA, RNA, peptide nucleic acids, phosphothioate-containing nucleic acids, phosphonate-containing nucleic acids and the like. There is no particular limit as to the number of units in a nucleic acid, provided that the nucleic acid contains 2 more nucleotides, nucleotide derivatives, or combinations thereof, specifically 5, 10, 15, 25, 50, 100, or more. Nucleic acids can encompass both single and double-stranded forms, and fully or partially duplex hybrids (e.g., RNA-DNA, RNA-PNA, or DNA-PNA).

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### EXAMPLE EMBODIMENTS

The present disclosure includes, but is not limited to, embodiments represented by the following clauses:

Clause 1: A composition comprising: a DNA polymerase; and an intercalating agent having the formula

X—Ar—R₁

wherein: X comprises an azide, a charged N, an amine, or an amino, wherein when X comprises an amino/amine, the amino/amine is NH₂, NR₂H, or NR₂R₃, wherein R₂ and R₃ are independently a DNA molecule to which the intercalating agent is covalently bonded or a group comprising an alkyl; Ar is a heterocyclic aromatic group with a ring structure having at least one charged N; and R₁ is attached to the charged N of the Ar and comprises an alkyl.

Clause 2: The composition of clause 1, wherein R₁ comprises a charged N, amine, and/or amino.

Clause 3: The composition of clause 2, wherein the charged N of R₁ is a quaternary ammonium.

Clause 4: The composition of clause 3, wherein the quaternary ammonium is attached to the Ar by a C₁-C₆ alkyl.

Clause 5: The composition of clause 4, wherein the quaternary ammonium is attached to the Ar by a C₂-C₃ alkyl.

Clause 6: The composition of any one of clauses 3-5, wherein the quaternary ammonium is bonded to three C₁-C₂ alkyl groups.

Clause 7: The composition of clause 6, wherein R₁ is:

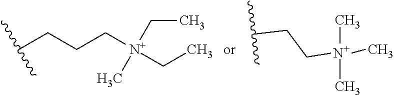

Clause 8: The composition of any one of clauses 1-7, wherein the ring structure of Ar has two or three rings.

Clause 9: The composition of clause 8, wherein Ar is a benzopyridine or dibenzopyridine.

Clause 10: The composition of clause 9, wherein Ar is a phenanthridine, quinoline, isoquinoline, acridine, or aminoacridine.

Clause 11: The composition of any one of clauses 1-10, wherein Ar is substituted with one or more substituents.

Clause 12: The composition of clause 11, wherein the substituents of the Ar are independently selected from the group consisting of phenyl, aromatic, halogen, alkyl, amine, amino, alkylamino, and dialkylamino.

Clause 13: The composition of clause 12, wherein at least one of the substituents is phenyl and/or at least one of the substituents is amino.

Clause 14: The composition of any one of clauses 1-13, wherein X is NH₃ or comprises a secondary or tertiary amine by which the intercalating agent is covalently bound to another molecule.

Clause 15: The composition of clause 14, wherein for at least some of the intercalating agent, X comprises a secondary or tertiary amine by which the intercalating agent is bound to DNA.

Clause 16: The composition of any one of clauses 14-15, wherein the intercalating agent comprises propidium (post-activated propidium monoazide).

Clause 17: The composition of any one of clauses 14-15, wherein the intercalating agent comprises ethidium (post-activated ethidium monoazide).

Clause 18: The composition of any one of clauses 14-15, wherein the intercalating agent comprises 6-amino-i-(3-(trimethylammonio)propyl)quinolin-1-ium (post-activated 6-azido-1-(3-(trimethylammonio)propyl)quinolin-1-ium).

Clause 19: The composition of any one of clauses 14-15, wherein the intercalating agent is included at a concentration of no more than about 100 μM, or no more than about 75 μM, or no more than about 50 μM, or no more than about 25 μM, no more than about 10 μM, or no more than about 7.5 μM, or no more than about 5 μM, or no more than about 2.5 μM, or no more than about 1 μM, or no more than about 750 nM, or no more than about 500 nM, or no more than about 250 nM, no more than about 100 nM, no more than about 75 nM, no more than about 50 nM, no more than about 25 nM, no more than about 10 nM, no more than about 7.5 nM, no more than about 5 nM, no more than about 2.5 nM, or no more than about 1 nM, or no more than about 0.1 nM.

Clause 20: The composition of any one of clauses 1-19, wherein the DNA polymerase is a thermostable DNA polymerase.

Clause 21: The composition of any one of clauses 1-20, wherein the DNA polymerase is Taq DNA polymerase, a mutant, variant, or derivative thereof.

Clause 22: The composition of any one of clauses 1-21, further comprising one or more nucleotides (dNTPs).

Clause 23: The composition of clause 22, wherein said nucleotides are selected from the group consisting of dTTP, dATP, dCTP, dGTP, 7-deaza-dGTP, or dUTP.

Clause 24: The composition of clause 23 or 24, wherein the concentration of each of said nucleotides is about 0.5 mM to 5 mM.

Clause 25: The composition of any one of clauses 1-24, further comprising one or more buffers and one or more salts.

Clause 26: The composition of any one of clauses 1-25, further comprising glycerol.

Clause 27: The composition of any one of clauses 1-26, further comprising an antifoam agent.

Clause 28: The composition of any one of clauses 1-27, further comprising an inhibitor tolerance agent.

Clause 29: The composition of clause 28, wherein the inhibitor tolerance agent includes one or more of gelatin, serum albumin, polyethylene glycol (PEG), resin, or agarose.

Clause 30: The composition of clause 29, wherein the gelatin is fish skin gelatin.

Clause 31: The composition of clause 29 or 30, wherein the inhibitor tolerance agent comprises bovine serum albumin.

Clause 32: The composition of any one of clauses 1-31, further comprising a polymerase antibody.

Clause 33: A kit comprising: a composition as in any one of clauses 1-32; and at least one pair of primers.

Clause 34: The kit of clause 33, further comprising one or more probes.

Clause 35: The kit of clause 33 or 34, wherein at least one primer and/or the probe include a detectable label.

Clause 36: A method of manufacturing a composition for use in amplification of nucleic acid, the method comprising: providing a first mixture that comprises one or more nucleic acid amplification components; adding to the first mixture an intercalating agent having the formula:

X—Ar—R₁

wherein: X comprises an azide, a charged N, an amine, or an amino; Ar is a heterocyclic aromatic group with a ring structure having at least one charged N; and R₁ is attached to the charged N of the Ar and comprises an alkyl; and exposing the first mixture to light to activate the intercalating agent and cause covalent bonding between the intercalating agent and contaminant DNA present in the first mixture.

Clause 37: The method of clause 36, wherein the one or more nucleic acid amplification components include a DNA polymerase.

Clause 38: The method of clause 36 or 37, wherein the one or more nucleic acid amplification components include a DNA polymerase antibody.

Clause 39: The method of any one of clauses 36-38, wherein the one or more nucleic acid amplification components include an inhibitor tolerance agent.

Clause 40: The method of clause 39, wherein the inhibitor tolerance agent is bovine serum albumin.

Clause 41: The method of any one of clauses 36-40, wherein the one or more nucleic acid amplification components are not amenable to autoclaving.

Clause 42: The method of any one of clauses 36-41, wherein the intercalating agent is added at a concentration of at least about 100 nM, or at least about 1 μM, or at least about 2 μM, or at least about 3 μM, or at least about 4 μM, at least about 5 μM, or at least about 10 μM, or at least about 20 μM, or at least about 40 μM, or at least about 60 μM, or at least about 80 μM, or at least about 100 μM, or at least about 120 μM, or at least about 140 μM, or at least about 160 μM, or at least about 180 μM, or at least about 200 μM, prior to exposing the first mixture to light to activate the intercalating agent.

Clause 43: The method of any one of clauses 36-42, wherein X comprises an azide for at least about 60%, or at least about 70%, or at least about 80%, or at least about 85%, or at least about 90%, or at least about 95%, or at least about 97% of the intercalating agent as added to the first mixture.

Clause 44: The method of any one of clauses 36-43, further comprising: providing a second mixture that comprises one or more nucleic acid amplification components; autoclaving the second mixture for at least 30 minutes; and combining the first mixture and the second mixture to form a combined mixture.

Clause 45: The method of clause 44, wherein the second mixture is autoclaved for at least 40 minutes, or at least 50 minutes, or at least 60 minutes, or at least 70 minutes, or at least 80 minutes, or at least 100 minutes, or at least 120 minutes, or at least 200 minutes.

Clause 46: The method of clause 44 or 45, wherein autoclaving is performed at a temperature of about 110° C. to about 130° C. and a pressure of about 80-120 kPa (about 12-17 psi).

Clause 47: The method of any one of clauses 44-46, wherein the one or more nucleic acid amplification components of the second mixture include one or more buffers and/or one or more salts.

Clause 48: The method of any one of clauses 44-47, wherein the one or more nucleic acid amplification components of the second mixture include glycerol and/or water.

Clause 49: The method of any one of clauses 44-48, wherein the one or more nucleic acid amplification components of the second mixture include an inhibitor tolerance agent.

Clause 50: The method of clause 49, wherein the inhibitor tolerance agent is gelatin.

Clause 51: The method of clause 50, wherein the gelatin is fish skin gelatin.

Clause 52: The method of any one of clauses 44-51, wherein the combined mixture corresponds to a composition as in any one of clauses 1-32.

Clause 53: The method of any one of clauses 36-52, wherein the intercalating bonds to the contaminant DNA via amine bonding.

Clause 54: The method of any one of clauses 36-53, wherein R₁ comprises a charged N, amine and/or amino.

Clause 55: The method of clause 54, wherein the charged N of R₁ is a quaternary ammonium.

Clause 56: The method of clause 55, wherein the quaternary ammonium is attached to the Ar by a C₁-C₆ alkyl.

Clause 57: The method of clause 55 or 56, wherein the quaternary ammonium is bonded to three C₁-C₂ alkyl groups.

Clause 58: The method of clause 57, wherein R₁ is:

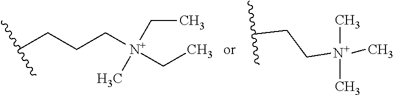

Clause 59: The method of any one of clauses 36-58, wherein the ring structure of Ar has two or three rings.

Clause 60: The method of clause 59, wherein Ar is a benzopyridine or dibenzopyridine.

Clause 61: The method of clause 60, wherein Ar is a phenanthridine, quinoline, isoquinoline, acridine, or aminoacridine.

Clause 62: The method of any one of clauses 36-61, wherein Ar is substituted with one or more substituents.

Clause 63: The method of clause 62, wherein the substituents of the Ar are independently selected from the group consisting of phenyl, aromatic, halogen, alkyl, amine, amino, alkylamino, and dialkylamino.

Clause 64: The method of clause 63, wherein at least one of the substituents is phenyl and/or at least one of the substituents is amino.

Clause 65: The method of any one of clauses 36-64, wherein X comprises an azide or is an azide.

Clause 66: The method of clause 65, wherein the intercalating agent comprises propidium monoazide.

Clause 67: The method of clause 65, wherein the intercalating agent is ethidium monoazide.

Clause 68: The method of any one of clauses 36-67, wherein the intercalating agent is 6-azido-1-(3-(trimethylammonio)propyl)quinolin-1-ium.

Clause 69: A method of manufacturing a composition for use in amplification of nucleic acid, the method comprising: providing a first mixture that comprises one or more nucleic acid amplification components; adding to the first mixture an intercalating agent having the formula:

X—Ar—R₁

wherein: X comprises an azide, a charged N, an amine, or an amino; Ar is a heterocyclic aromatic group with a ring structure having at least one charged N; and R₁ is attached to the charged N of the Ar and comprises an alkyl and optionally a charged N; exposing the first mixture to light to activate the intercalating agent and cause covalent bonding between the intercalating agent and contaminant DNA present in the first mixture; providing a second mixture that comprises one or more nucleic acid amplification components; autoclaving the second mixture for at least 30 minutes; and combining the first mixture and the second mixture to form a combined mixture.

Clause 70: The method of clause 69, wherein the first mixture comprises one or more of a DNA polymerase, a DNA polymerase antibody, or bovine serum albumin.

Clause 71: The method of clause 69 or 70, wherein the second mixture comprises one or more of a buffer, a salt, glycerol, an antifoam agent, or gelatin.

Clause 72: The method of any one of clauses 69-71, wherein X comprises an azide or is an azide.

Clause 73: A method of amplifying a nucleic acid, comprising: providing a composition as in any one of clauses 1-32; mixing the composition with a pair of primers; and subjecting the composition and primers to amplification conditions to enable amplification of the target nucleic acid.

Clause 74: The method of clause 73, wherein the amplification is a polymerase chain reaction (PCR).

Clause 75: The method of clause 73, wherein the PCR is quantitative PCR (qPCR).

Clause 76: The method of clause 73, wherein the PCR is digital PCR (dPCR).

Clause 77: The method of any one of clauses 73-76, further comprising subjecting the target nucleic acid to a reverse transcription reaction prior to amplification via PCR.

### EXAMPLES

### Example 1

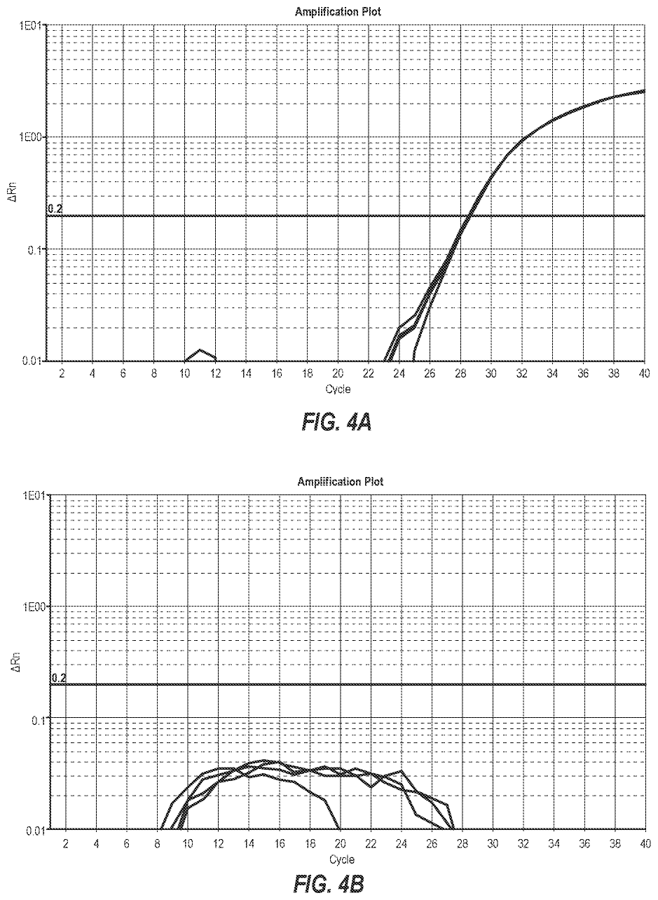

A pan-bacterial (16S) assay was performed to compare amplification of the NTC of a conventional master mix to amplification of the NTC of the master mix composition prepared according to some embodiment. FIGS. 4A and 4B illustrate qPCR amplification plots showing the results. As shown, the NTC of the conventional master mix (FIG. 4A) showed higher background signal as compared to the NTC of the presently disclosed master mix (FIG. 4B).

### Example 2

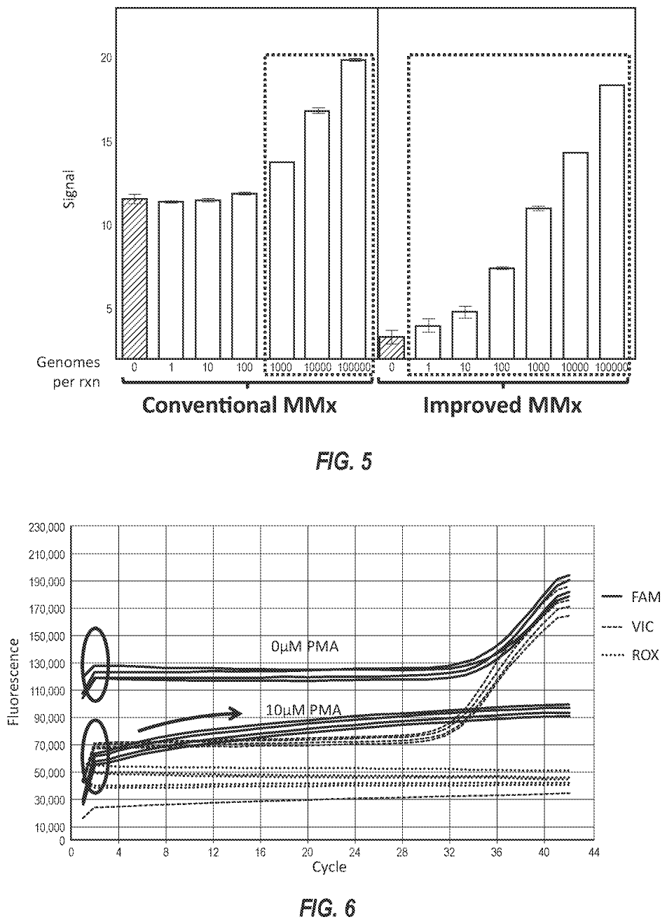

A pan-bacterial (16S) assay was performed to compare the amplification signal of both a conventional master mix and the master mix prepared according to some embodiments over various amounts of spiked-in bacterial genomes (0, 1, 10, 100, 1,000, 10,000, and 100,000 genomes per reaction). As shown by FIG. 5, the conventional master mix obscured any signal beneath the contamination level, whereas the master mix according to the present disclosure enabled a lower LOD and an expanded dynamic range.

### Example 3

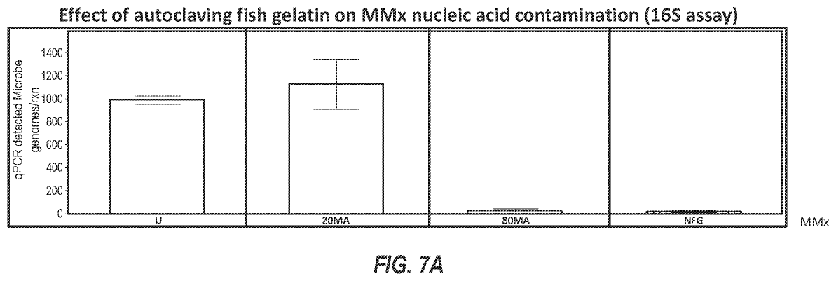

Amplification was carried out using different detectable labels and different amounts of added PMA. FIG. 6 illustrates the fluorescence signals of various dyes during/resulting from the amplification, showing that the master mix composition including 10 μM of PMA reduced the FAM signal as compared to an otherwise similar master mix composition lacking PMA. This illustrates that it may be preferable to limit the amount of intercalating agent used in the final master mix composition.

### Example 4

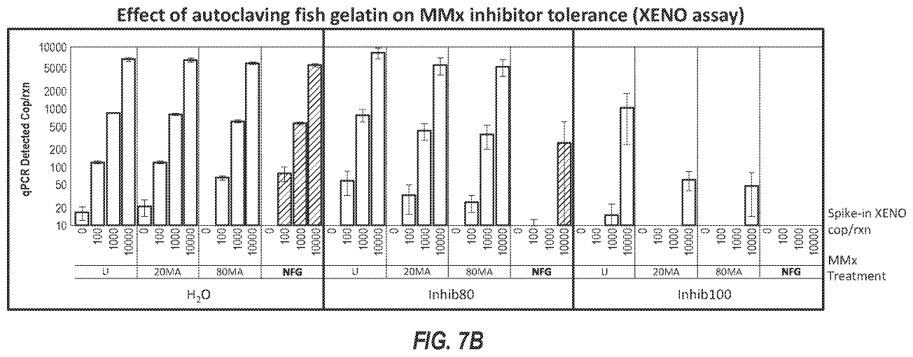

Fish skin gelatin was autoclaved according to a standard protocol (“20MA”=20 min.) and an extended protocol (“80MA”=80 min.). The fish skin gelatin was then utilized in a pan-bacterial (16S) qPCR assay. FIG. 7A illustrates the results, showing that the pan-bacterial (16S) qPCR assay detected similar levels of contaminant DNA in the master mix composition with untreated fish gelatin (“U”=fish skin gelatin, no autoclaving) and the composition with fish gelatin autoclaved for 20 minutes, and showing substantial lack of contamination in the negative control (“NFG”=master mix composition with no fish skin gelatin) and the 80MA composition with fish gelatin autoclaved for 80 minutes. The extended autoclaving protocol thus significantly reduced or even eliminated contaminant DNA associated with the fish skin gelatin.

### Example 5

Fish skin gelatin autoclaved according to the standard or extended protocol as in Example 4 was utilized in master mix compositions that were spiked with varying known amounts of a XENO sequence and varying amounts of a PCR inhibitor (humic acid). The aim was to determine what effect, if any, the extended autoclaving protocol had on the ability of the fish skin gelatin to provide inhibitor tolerance when used in a master mix. In FIG. 7B, the “H₂O” treatment did not include humic acid, the “Inhib80” treatment included humic acid at a concentration of 80 ng/μl, and the “Inhib100” treatment included humic acid at a concentration of 100 ng/μl. The U, 20MA, 80MA, and NFG labels represent the same as in Example 4 and FIG. 7A.

FIG. 7B illustrates that at a concentration of 80 ng/μl of a humic acid inhibitor, the composition receiving the extended autoclaved fish gelatin performed substantially the same as the composition receiving the standard autoclaved fish gelatin and the control composition receiving no fish gelatin, and showing that at a concentration of 100 ng/μl of humic acid, the composition receiving the extended autoclaved fish gelatin performed substantially the same as the composition receiving the standard autoclaved fish gelatin. These results illustrate that extended autoclaving did not detrimentally affect the ability of the inhibitor tolerance agent to function as intended.

### Example 6

The master mix composition described herein was utilized in various quality control screening panels designed to detect contaminant DNA. The assays were formulated for detecting E. coli, fungal (pan), bacterial (pan, 16S rRNA gene), 18S rRNA gene, splicing factor SRi 1, kanamycin resistance genes, beta-lactamase, the mecA gene, and the vanA gene. In some embodiments, the master mix can be utilized to detect various contaminant DNA including, but not limited to human herpesvirus 5, cytomegalovirus and adenovirus.

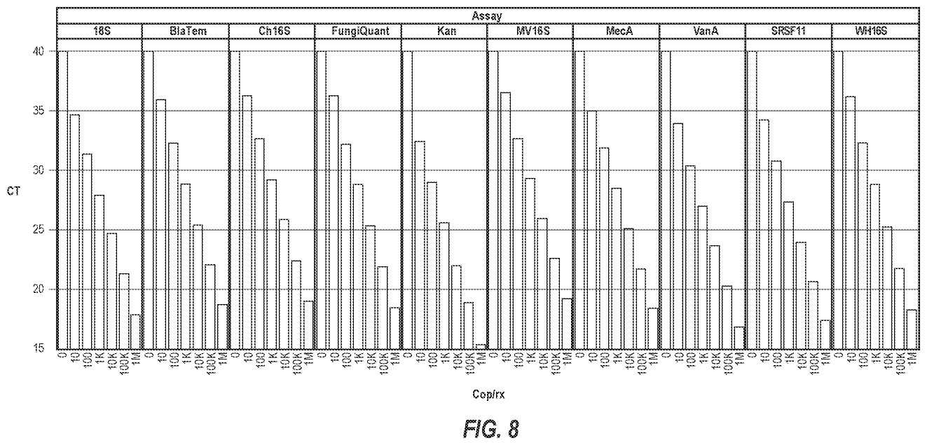

FIG. 8 illustrates the results of a quality control screening panel, showing that for all qPCR assays tested, the master mix compositions according to some embodiments performed as expected for known amounts of spiked-in nucleic acid and showed no detectable contamination.

1. A composition comprising:
a DNA polymerase; and
an intercalating agent having the formula
X—Ar—R₁
wherein
X comprises an azide, a charged N, an amine, or an amino, wherein when X comprises an amino/amine, the amino/amine is NH₂, NR₂H, or NR₂R₃, wherein R₂ and R₃ are independently a DNA molecule to which the intercalating agent is covalently bonded or a group comprising an alkyl,
Ar is a heterocyclic aromatic group with a ring structure having at least one charged N, and
R₁ is attached to the charged N of the Ar and comprises an alkyl.

2. The composition of claim 1, wherein R₁ comprises a charged N, amine, and/or amino.

3. The composition of claim 2, wherein the charged N of R₁ is a quaternary ammonium.

4. The composition of claim 3, wherein the quaternary ammonium is attached to the Ar by a C₁-C₆ alkyl.

5. The composition of claim 4, wherein the quaternary ammonium is attached to the Ar by a C₂-C₃ alkyl.

6. The composition of claim 3, wherein the quaternary ammonium is bonded to three C₁-C₂ alkyl groups.

7. The composition of claim 6, wherein R₁ is:

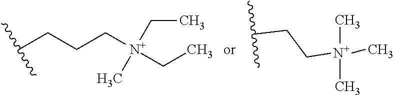

8. The composition of claim 1, wherein the ring structure of Ar has two or three rings.

9. The composition of claim 8, wherein Ar is a benzopyridine or dibenzopyridine.

10. The composition of claim 9, wherein Ar is a phenanthridine, quinoline, isoquinoline, acridine, or aminoacridine.

11. The composition of claim 1, wherein Ar is substituted with one or more substituents.

12. The composition of claim 11, wherein the substituents of the Ar are independently selected from the group consisting of phenyl, aromatic, halogen, alkyl, amine, amino, alkylamino, and dialkylamino.

13. The composition of claim 12, wherein at least one of the substituents is phenyl and/or at least one of the substituents is amino.

14. The composition of claim 1, wherein X is NH₃ or comprises a secondary or tertiary amine by which the intercalating agent is covalently bound to another molecule.

15. The composition of claim 14, wherein for at least some of the intercalating agent, X comprises a secondary or tertiary amine by which the intercalating agent is bound to DNA.

16. The composition of claim 14, wherein the intercalating agent comprises propidium (post-activated propidium monoazide).

17. The composition of claim 14, wherein the intercalating agent comprises ethidium (post-activated ethidium monoazide).

18. The composition of claim 14, wherein the intercalating agent comprises 6-amino-1-(3-(trimethylammonio)propyl)quinolin-1-ium (post-activated 6-azido-1-(3-(trimethylammonio)propyl)quinolin-1-ium).

19. The composition of claim 14, wherein the intercalating agent is included at a concentration of no more than about 100 μM, or no more than about 75 μM, or no more than about 50 μM, or no more than about 25 μM, no more than about 10 μM, or no more than about 7.5 μM, or no more than about 5 μM, or no more than about 2.5 μM, or no more than about 1 μM, or no more than about 750 nM, or no more than about 500 nM, or no more than about 250 nM, no more than about 100 nM, no more than about 75 nM, no more than about 50 nM, no more than about 25 nM, no more than about 10 nM, no more than about 7.5 nM, no more than about 5 nM, no more than about 2.5 nM, or no more than about 1 nM, or no more than about 0.1 nM.

20. The composition of claim 1, wherein the DNA polymerase is a thermostable DNA polymerase.

21. The composition of claim 1, wherein the DNA polymerase is Taq DNA polymerase, a mutant, variant, or derivative thereof.

22. The composition of claim 1, further comprising one or more nucleotides (dNTPs).

23. The composition of claim 22, wherein said nucleotides are selected from the group consisting of dTTP, dATP, dCTP, dGTP, 7-deaza-dGTP, or dUTP.

24. The composition of claim 23, wherein the concentration of each of said nucleotides is about 0.5 mM to 5 mM.

25. The composition of claim 1, further comprising one or more buffers and one or more salts.

26. The composition of claim 1, further comprising glycerol.

27. The composition of claim 1, further comprising an antifoam agent.

28. The composition of claim 1, further comprising an inhibitor tolerance agent.

29. The composition of claim 28, wherein the inhibitor tolerance agent includes one or more of gelatin, serum albumin, polyethylene glycol (PEG), resin, or agarose.

30. The composition of claim 29, wherein the gelatin is fish skin gelatin.

31. The composition of claim 29, wherein the inhibitor tolerance agent comprises bovine serum albumin.

32. The composition of claim 1, further comprising a polymerase antibody.

33. A kit comprising:
a composition as in any one of claims 1-32; and
at least one pair of primers.

34. The kit of claim 33, further comprising one or more probes.

35. The kit of claim 33, wherein at least one primer and/or the probe include a detectable label.

36. A method of manufacturing a composition for use in amplification of nucleic acid, the method comprising:
providing a first mixture that comprises one or more nucleic acid amplification components;
adding to the first mixture an intercalating agent having the formula:
X—Ar—R₁
wherein
X comprises an azide, a charged N, an amine, or an amino,
Ar is a heterocyclic aromatic group with a ring structure having at least one charged N, and
R₁ is attached to the charged N of the Ar and comprises an alkyl; and
exposing the first mixture to light to activate the intercalating agent and cause covalent bonding between the intercalating agent and contaminant DNA present in the first mixture.

37. The method of claim 36, wherein the one or more nucleic acid amplification components include a DNA polymerase.

38. The method of claim 36, wherein the one or more nucleic acid amplification components include a DNA polymerase antibody.

39. The method of claim 36, wherein the one or more nucleic acid amplification components include an inhibitor tolerance agent.

40. The method of claim 39, wherein the inhibitor tolerance agent is bovine serum albumin.

41. The method of claim 36, wherein the one or more nucleic acid amplification components are not amenable to autoclaving.

42. The method of claim 36, wherein the intercalating agent is added at a concentration of at least about 100 nM, or at least about 1 μM, or at least about 2 μM, or at least about 3 μM, or at least about 4 μM, at least about 5 μM, or at least about 10 μM, or at least about 20 μM, or at least about 40 μM, or at least about 60 μM, or at least about 80 μM, or at least about 100 μM, or at least about 120 μM, or at least about 140 μM, or at least about 160 μM, or at least about 180 μM, or at least about 200 μM, prior to exposing the first mixture to light to activate the intercalating agent.

43. The method of claim 36, wherein X comprises an azide for at least about 60%, or at least about 70%, or at least about 80%, or at least about 85%, or at least about 90%, or at least about 95%, or at least about 97% of the intercalating agent as added to the first mixture.

44. The method of claim 36, further comprising:
providing a second mixture that comprises one or more nucleic acid amplification components;
autoclaving the second mixture for at least 30 minutes; and
combining the first mixture and the second mixture to form a combined mixture.

45. The method of claim 44, wherein the second mixture is autoclaved for at least 40 minutes, or at least 50 minutes, or at least 60 minutes, or at least 70 minutes, or at least 80 minutes, or at least 100 minutes, or at least 120 minutes, or at least about 200 minutes.

46. The method of claim 44, wherein autoclaving is performed at a temperature of about 110° C. to about 130° C. and a pressure of about 80-120 kPa (about 12-17 psi).

47. The method of claim 44, wherein the one or more nucleic acid amplification components of the second mixture include one or more buffers and/or one or more salts.

48. The method of claim 44, wherein the one or more nucleic acid amplification components of the second mixture include glycerol and/or water.

49. The method of claim 44, wherein the one or more nucleic acid amplification components of the second mixture include an inhibitor tolerance agent.

50. The method of claim 49, wherein the inhibitor tolerance agent is gelatin.

51. The method of claim 50, wherein the gelatin is fish skin gelatin.

52. The method of claim 44, wherein the combined mixture corresponds to a composition as in any one of claims 1-32.

53. The method of claim 36, wherein the intercalating bonds to the contaminant DNA via amine bonding.

54. The method of claim 36, wherein R₁ comprises a charged N, amine and/or amino.

55. The method of claim 54, wherein the charged N of R₁ is a quaternary ammonium.

56. The method of claim 55, wherein the quaternary ammonium is attached to the Ar by a C₁-C₆ alkyl.

57. The method of claim 55, wherein the quaternary ammonium is bonded to three C₁-C₂ alkyl groups.

58. The method of claim 57, wherein R₁ is:

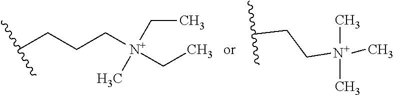

59. The method of claim 36, wherein the ring structure of Ar has two or three rings.

60. The method of claim 59, wherein Ar is a benzopyridine or dibenzopyridine.

61. The method of claim 60, wherein Ar is a phenanthridine, quinoline, isoquinoline, acridine, or aminoacridine.

62. The method of claim 36, wherein Ar is substituted with one or more substituents.

63. The method of claim 62, wherein the substituents of the Ar are independently selected from the group consisting of phenyl, aromatic, halogen, alkyl, amine, amino, alkylamino, and dialkylamino.

64. The method of claim 63, wherein at least one of the substituents is phenyl and/or at least one of the substituents is amino.

65. The method of claim 36, wherein X comprises an azide or is an azide.

66. The method of claim 65, wherein the intercalating agent comprises propidium monoazide.

67. The method of claim 65, wherein the intercalating agent is ethidium monoazide.

68. The method of claim 36, wherein the intercalating agent is 6-azido-1-(3-(trimethylammonio)propyl)quinolin-1-ium.

69. A method of manufacturing a composition for use in amplification of nucleic acid, the method comprising:
providing a first mixture that comprises one or more nucleic acid amplification components;
adding to the first mixture an intercalating agent having the formula:
X—Ar—R₁
wherein
X comprises an azide, a charged N, an amine, or an amino,
Ar is a heterocyclic aromatic group with a ring structure having at least one charged N, and
R₁ is attached to the charged N of the Ar and comprises an alkyl and optionally a charged N;
exposing the first mixture to light to activate the intercalating agent and cause covalent bonding between the intercalating agent and contaminant DNA present in the first mixture;
providing a second mixture that comprises one or more nucleic acid amplification components;
autoclaving the second mixture for at least 30 minutes; and
combining the first mixture and the second mixture to form a combined mixture.

70. The method of claim 69, wherein the first mixture comprises one or more of a DNA polymerase, a DNA polymerase antibody, or bovine serum albumin.

71. The method of claim 69, wherein the second mixture comprises one or more of a buffer, a salt, glycerol, an antifoam agent, or gelatin.

72. The method of claim 69, wherein X comprises an azide or is an azide.

73. A method of amplifying a nucleic acid, comprising:
providing a composition as in any one of claims 1-32;
mixing the composition with a pair of primers; and
subjecting the composition and primers to amplification conditions to enable amplification of the target nucleic acid.

74. The method of claim 73, wherein the amplification is a polymerase chain reaction (PCR).

75. The method of claim 73, wherein the PCR is quantitative PCR (qPCR).

76. The method of claim 73, wherein the PCR is digital PCR (dPCR).

77. The method of claim 73, further comprising subjecting the target nucleic acid to a reverse transcription reaction prior to amplification via PCR.
